(19) United States
(12) Patent Application Publication
(10) Pub. No.: US 2012/0066875 A1
(43) Pub. Date: Mar. 22, 2012

(54) METHOD AND SYSTEM FOR GRADING BEDDING FABRIC

(76) Inventor: Gerald E. Wootten, JR., Cullman, AL (US)

(21) Appl. No.: 12/883,673

(22) Filed: Sep. 16, 2010

(51) Int. Cl. G01N 33/36

(52) U.S. Cl. 26/1; 26/70; 73/159; 705/500

(57) ABSTRACT

A method for grading a bedding fabric comprises selecting at least two grading parameters and assigning a grade to the fabric based upon the selected grading parameters in order to grade the fabric between a minimum grade and a maximum grade. The grade is designed to assist a consumer in making consistent purchases. The grading parameters may include cost, type of fabric, thread count, comfort performance characteristics, ply, weight, fiber density and/or thickness of the fabric, aesthetic appearance of the fabric, durability, weave, bacterial, allergen, and/or fungal resistant properties, and the eco-friendly nature of the fabric. A grading system for a packaged bedding fabric includes a packaged bedding product and a grade coupled to the packaged bedding product such that a consumer can determine the quality of the bedding product based upon the grade.

### FIELD

The technology described herein relates to a method for grading bedding fabric. The method could be used with any type of fabric that a consumer would regularly purchase.

### BACKGROUND

The conventional system for grading bedding fabric has been via thread count. Thread count is the number of threads, both vertical and horizontal, found in a one-inch fabric square of the product. Consumers often assume that a higher thread count fabric is better than a lower thread count fabric. The notion that high thread count is better, softer and more luxurious has influenced many shoppers to insist it is the only way to go when purchasing sheets for the bedroom. Unfortunately, thread count is unreliable, misunderstood, and not trustworthy as a predictor of quality of bedding fabrics. Thread count has become more of a marketing ploy than a true predictor of quality. High thread count fabrics often provide an “illusion” of value, which can result in consumer disappointment and confusion. Thread count simply does not tell the whole story of quality and value when considering purchasing bedding, such as sheets. It is desirable to minimize the impact of thread count in grading fabric.

### SUMMARY

In accordance with the teachings described herein, a method for grading bedding fabric is described.

### BRIEF DESCRIPTION OF THE DRAWING FIGURES

FIG. 1 is a perspective view of a fabric display presenting different fabric panels for consideration to a consumer, with each fabric panel having an associated grade.

### DETAILED DESCRIPTION

The technology described herein relates to a method for grading fabric where thread count can be considered, but is not the only indicator for quality of the fabric. The method is designed to provide a reliable predictor of bedding quality and value that can be trusted by the consumer. The method involves establishing “degrees,” or a grading scale. One preferred grading scale is 1 degree through 7 degrees, with 7 degrees being the highest grade and 1 degree being the lowest grade. Alternatively, 1 could be the highest grade and 7 could be the lowest grade, or more than 7 grades could be used. We periodically utilize the terminology “degrees” herein to describe the grading, but other terminology could also be used. Letters or symbols could be used instead of numbers. For example, stars could be used, with one star being lower grade and 5 stars being higher grade. Alternatively, colors or color schemes could be used, such as lighter shades for lower grades and darker shades for higher grades, or vice versa. Thus, the example grading system discussed herein is not limited to a particular word, number, symbol, or color scheme.

The degree system described herein offers a different degree of fit, feel, function, and fashion with innovative, meaningful, and lasting benefits. This system, in turn, creates a different ownership experience for the consumer, which assists in creating long-term customer loyalty.

The degree system introduces a new level of comfort, fashion, and value based upon the type of fabric, the weight of the fabric, and the comfort performance characteristics of the fabric. Comfort performance type characteristics include terms like softness, crispness, dryness, and silkiness. These terms also describe what is known as the “hand” of the fabric, which is the way the fabric feels when touched.

The thickness and ply of the threads also plays a role in the smoothness and softness of the material. The number of threads entwined in a single thread make up the ply. The two common types of ply found in most sheets are single-ply and two-ply. Single-ply fabrics use one thread while two-ply fabrics use two threads that are entwined. The ply of the fabric may mean the difference between a stated 250 and 500 thread count, with the 250 coming from single-ply and the 500 coming from two-ply. A higher thread count is typically more resistant to pilling. Pilling is a tangled ball of fibers that appears on the surface of a fabric, as a result of wear or continued friction or rubbing on the surface of the fabric.

Thread thickness and quality are two factors that determine the ultimate feel of the bedding. Finer threads made of high quality strands typically create a soft, smooth and delicate sheet fabric. In fact, a 250 thread count sheet made of premium thread can be much more luxurious than a 500 count sheet with a lower quality thread. A thread count over 500 typically is composed of two-ply yarns and a thread count 400 and below is typically constructed of single-ply yarns. Two-ply yarns are often softer, sleeker, and more luxurious. A bigger yarn size is often better and means a more tightly woven and quality sheet. The standard range for an acceptable sheet set is yarn weight between 4 and 100. The higher the yarn size, the finer the yarn and the stronger and softer the material. Finer yarns allow for lighter, more supple fabrics.

Thread weave is another contributing factor in fabric quality. Some common weaves found in bed sheets include flannel, jersey, percale, and sateen. Flannel is typically made of cotton and has a soft, napped finish. It is of a medium weight plain or twill woven fabric. Jersey is an elastic material that is a knitted fabric with a plain stitch and has good every-day wear and draping characteristics. Percale is made from cotton that is combed and carded. It is a plain closely woven fabric that has a smooth, silky texture and feel. It typically is of medium weight and low to medium thread count (180 to 250 threads per square inch). Carding is the disentanglement, cleaning and intermixing of fibers to produce a continuous web or sliver suitable for subsequent processing. This is achieved by passing the fibers between moving pins, wires or teeth. The combing process is an additional step beyond carding. In this process, the fibers are arranged in a highly parallel form, and additional short fibers are removed, producing high quality yarns with excellent strength, fineness, and uniformity. Sateen is softer and has a shiny appearance. It is made of a weave that provides more yarn surface on the clothes' face. A sateen wave is produced by floating fill yarns over warp yarns.

Fabrics may be mercerized, giving the fabric greater luster and strength. Mercerization is a treatment of yarns or fabrics with caustic alkali, in which fibers are swollen and stretched. Some bedding fabrics are Sanforized®, a process designed to help reduce shrinkage.

Table 1 below identifies different types of fabric that correspond with different degrees within the example method. As is evident, thread count only plays a part in the overall grading system.

TABLE 1

| Degree | Material | Thread Count |
|---|---|---|
| 1^{st} | Basic Cotton-Rich | 250 |
| 2^{nd} | Choice Natural Cotton | 260 |
| 3^{rd} | Select World Class Cotton | 300 |
| 4^{th} | Preferred Egyptian Cotton | 400 |
| 5^{th} | Premium Bamboo-Rich | 300 |
| 6^{th} | Superior Tencel | 300 |
| 6^{th} | American Supima Cotton | 600 |
| 7^{th} | Elite American Supima Cotton | 1020 |

A brief explanation of each of the above categories follows. In addition, more categories than those noted may be utilized, if desired.

7^{th} Degree: This is an elite grade. This category of bedding may include 100% American Supima cotton. It may include hem adornments, such as a euro-pleat, as well as distinctive hem-stitch treatments. The 7^{th} degree bedding has elite weight and crispness compared to other fabrics. It features a 1020 single ply, authentic thread count crafted from Supima extra long staple length (ELS) cotton. By United States law, an ELS cotton must have a fiber length of 1⅜ inches or longer. The majority of Supima cotton is 1½ inch staple. Supima is the worlds' finest cotton. Supima is short for Superior Pima and is known for its superior fiber length, strength, and natural luster. ELS accounts for only 3% of the total world cotton production. Fiber length directly translates into optimum performance for fabrics. Supima's extra length creates softer, finer fabrics that have a more supple drape. The length also creates a more lustrous fabric surface that is less prone to pilling. In addition, Supima's uniformity in fiber density and weight allows the fabric to absorb a dye at a more even rate that provides more lasting and vibrant colors. Supima's strength adds durability and the ability to withstand the application of performance finishes, such as non-wrinkle and anti-microbial, that can significantly weaken fabric strength

6^{th} Degree: This is considered a superior grade: This category of bedding may be 100% American Supima cotton. It may include a distinctive hem stitching, such as the triple baratto stitch hem. The fabric has superior weight and crispness and is a luxurious sateen weave for softness. It features a 600 single ply, authentic thread count.

The 6^{th} degree fabric may also include a 100% Tencel performance fiber. Tencel provides a smooth, silky softness and texture that is resistant to bacteria and allergens. Tencel can withstand repeated washings without pilling or fiber breakage, resulting in a longer life. Tencel is also temperature regulating for optimal comfort, with a “cool in summer, warm in winter” feel. Tencel also has a moisture management attribute that draws and releases moisture from the body. It is 70% more absorbent than cotton, but channels the moisture away resulting in a drier, more comfortable sleeping environment. Tencel is inherently bacteriostatic inhibiting the growth of bacteria without using harmful chemicals. Tencel is a performance fiber that can be made from the eucalyptus tree or other trees. The 6^{th} degree may also include a tencel quilted sheet ensemble, where the top sheet and pillow cases are quilted on one side for warmth without weight. This grade of fabric may be 300 authentic thread count. Tencel is also known as Lyocell. It has a soft finish and packs light.

5^{th} Degree. This is considered a premium grade. This category of bedding may be is made from premium bamboo rich fibers. Bamboo linens are extremely soft and naturally resistant to pilling and shrinkage. Bamboo is moisture wicking and provides ultra soft comfort and breathability. Wickability is the ability of a fiber or fabric to disperse moisture and allow it to pass through to the surface of the fabric, so that evaporation can take place. Breathability is the ability of a fabric, coating or laminate to transfer water vapor from one of its surfaces through the material to the other surface. Bamboo is naturally hypo-allergenic, and no pesticides are used because of its natural bacterial resistance. Bamboo will not hold odors and dries twice as fast as most fabrics. Bamboo is the most sustainable of all natural fibers, and is 100% biodegradable when returned to the earth. The 5^{th} degree may also include a bamboo quilted sheet ensemble, where the top sheet and pillow cases are quilted on one side for warmth without weight.

The 5^{th} degree category may also include a premium Italian memory pillow, which includes plush-firm support. This type of pillow is responsive to personalized comfort, includes premium Italian memory foam, and has a removable and washable cover.

The 5^{th} degree category may also include a premium, waterproof, enhanced performance bamboo mattress pad. The pad can include a bamboo-cotton fabric. This type of material is allergen resistant and can include a Fosshield®-type fiber for bacteria and fungus resistance.

4^{th} Degree. This is considered a preferred grade. This category of bedding material is made from preferred 100% Extra Long Staple Egyptian cotton. It has silky, smooth, lasting comfort and a light texture and weight. It may include a 400 thread count.

3^{rd} Degree. This is considered a select grade. This category of bedding material is made from select world-class cotton, such as 100% select long staple cotton for luxurious comfort. The comfort remains after repeated washing and drying of the fabric. This category could include a select comfort protection value pack, which includes a set of sheets and a basic mattress protector combination. The mattress protector could be a terry protector.

2^{nd} Degree. This is considered a choice grade. This category of bedding material is made from 100% choice natural cotton. This type of cotton is ultra soft. It may be a combed cotton, which has extra yarn refining to assure a luxurious feel, and a sateen weave. This category offering may include a choice comfort protection value pack, which could include a choice sheet set and a basic mattress protector combo, such as a terry protector.

1^{st} Degree. This is considered a basic grade. This category of bedding material is made from a basic, cotton rich blend, such as a 60% cotton, 40% polyester blend, or a 50/50 polyester/cotton blend. A polyester and cotton blend provides extended product life and is wrinkle resistant. The first degree could be a 250 thread count or a 200 thread count or another thread count, if desired. A 100% cotton terry waterproof mattress protection could also fall within this category and includes a waterproof barrier that protects the mattress, but that is breathable for sleeping comfort. This category may also include a basic comfort protection value pack, which can include a basic cotton sheet set and a terry mattress protector. In addition, the 1^{st} degree can include a promotional poly-filled pillow that includes 100% polyester fill and is hypoallergenic. This can include specially shaped pillows, such as Euro inserts for Euro shams, or conventionally sized pillow offerings, such as standard, queen, and king.

The above-discussion of grading is for exemplary purposes and could include additional materials and offerings. A number of factors go into determining what types of materials are categorized in different grades. These factors include the cost of the fabric, the type of fabric, the thread count of the fabric, the comfort performance characteristics of the fabric, the ply of the fabric, the weight of the fabric, the fiber density and/or thickness of the fabric, the aesthetic appearance of the fabric, the durability of the fabric, the weave of the fabric, the bacterial, allergen, and/or fungus resistant properties of the fabric, and the eco-friendly nature of the fabric. The grade may be established by considering two or more of these factors. Thus, the rating scale is somewhat subjective and subject to modification based upon new product offerings. However, the scale provides to the consumer a gauge for judging the quality of the bedding material on a consistent basis, with 7^{th} degree being the highest overall quality and 1^{st} degree being the lowest overall quality of materials. The consumer may find that they prefer a grade 4 over a higher grade and then may consistently select the grade that is right for them.

The comfort performance characteristics of the fabric include one or more of softness, crispness, temperature regulating characteristics, and moisture wicking characteristics. The eco-friendly nature of the fabric includes at least one of biodegradability, manufacturing techniques utilized to manufacture the fabric, and sustainability of the natural fibers.

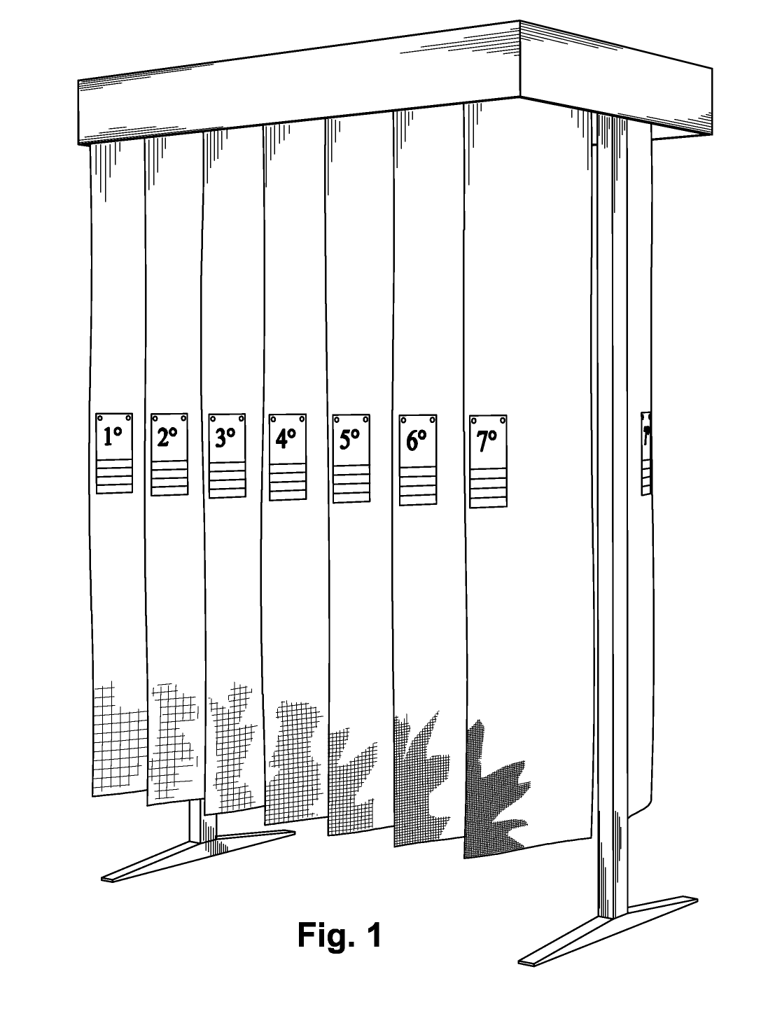

FIG. 1 depicts a series of fabric swatches that are hanging from an interactive display. As shown, each fabric swatch includes a label that identifies the grade of the fabric. The grades range from 1 degree to 7 degrees. Packaged bedding may also be utilized, such as a sheet set or comforter set that is packaged in a single unit. The grade of the bedding should be prominently displayed so that a consumer can make an informed purchasing decision. In selecting a grade of fabric, the consumer can test out the fabric using the display and then select a grade of fabric like that displayed in order to make an informed purchasing decision.

A grading system for a packaged bedding product may include a packaged bedding product and a grade associated with each product. The grade is coupled to the packaged bedding product such that a consumer can visibly determine the quality of the bedding product in order to make consistent bedding purchases without even having to feel the fabric. The grade may be established using the above-described parameters. A consumer who makes an initial purchase who likes the quality of the purchase can then make subsequent purchases by utilizing the grade that was assigned to the previous purchase. In this manner, a consumer can make more consistent decisions concerning bedding, rather than relying solely on thread count.

The term “substantially,” if used herein, is a term of estimation.

While various features are presented above, it should be understood that the features may be used singly or in any combination thereof. Further, it should be understood that variations and modifications may occur to those skilled in the art to which the claimed examples pertain. The examples described herein are exemplary. The disclosure may enable those skilled in the art to make and use alternative designs having alternative elements that likewise correspond to the elements recited in the claims. The intended scope may thus include other examples that do not differ or that insubstantially differ from the literal language of the claims. The scope of the disclosure is accordingly defined as set forth in the appended claims.

What is claimed is:

1. A method for grading a bedding fabric comprising:
selecting at least two grading parameters including:
the cost of the fabric,
the type of fabric,
the thread count of the fabric,
the comfort performance characteristics of the fabric,
the ply of the fabric,
the weight of the fabric,
the fiber density and/or thickness of the fabric,
the aesthetic appearance of the fabric,
the durability of the fabric,
the weave of the fabric,
the bacterial, allergen, and/or fungus resistant properties of the fabric, and
the eco-friendly nature of the fabric; and
assigning a grade to the fabric based upon the selected grading parameters in order to grade the fabric between a minimum grade and a maximum grade.

2. The method of claim 1, further comprising applying the grade to packaging that is associated with the fabric.

3. The method of claim 1, wherein the comfort of the fabric includes one or more of softness, crispness, temperature regulating characteristics, and moisture wicking characteristics.

4. The method of claim 1, wherein the eco-friendly nature of the fabric includes at least one of biodegradability, manufacturing techniques utilized to manufacture the fabric, and sustainability of the natural fibers.

5. The method of claim 1, wherein the thread count is an authentic thread count.

6. The method of claim 1, wherein the minimum grade is the lowest quality fabric and the maximum grade is the highest quality fabric, and further comprising intermediate grades between the minimum grade and the maximum grade, with intermediate grades corresponding to quality levels between minimum and maximum.

7. The method of claim 1, wherein the grade is in degrees that range from 1 degree to 7 degrees, with 7 degrees representing the highest quality fabric and 1 degree representing the lowest quality fabric.

8. The method of claim 1, wherein the grade is a numbering system that ranges from a minimum number to a maximum number, with the minimum number corresponding to a lower quality and the maximum number corresponding to a higher quality.

9. The method of claim 1, wherein the grade is a numbering system that ranges from a minimum number to a maximum number, with the maximum number corresponding to a lower quality and the minimum number corresponding to a higher quality.

10. The method of claim 1, wherein the grade is a lettering system that ranges from a first letter to a different letter.

11. The method of claim 1, wherein the type of fabrics, ranging from lowest quality to highest quality, includes 60/40 blend cotton, 100% natural cotton, 100% long staple cotton, 100% Egyptian extra long staple cotton, bamboo/cotton blend, tencel, and supima cotton.

12. The method of claim 1, wherein the thread count of the fabric ranges from about a 180 authentic thread count at the lowest quality to about a 1020 authentic thread count at the highest quality.

13. The method of claim 1, wherein the durability of the fabric includes at least one of resistance to pilling, withstanding repeated washing and drying, and withstanding the application of performance finishes that can weaken fabric strength.

14. A grading system for a packaged bedding product comprising:
a packaged bedding product; and
a grade coupled to the packaged bedding product such that a consumer can determine the quality of the bedding product based upon the grade, wherein the grade is established utilizing the method of claim 1.

15. The grading system of claim 14, wherein the packaged bedding product includes an advertising panel that identifies the bedding product and the grade, with the grade being visible to a consumer to assist in a purchasing decision.

16. The grading system of claim 14, wherein the grade is a number that ranges from 1 to 7, with 1 being the lowest quality and 7 being the highest quality.

17. The grading system of claim 14, wherein the grade is one of a letter, a number, a symbol, or a color.
